(19) United States
(12) Patent Application Publication
(10) Pub. No.: US 2009/0060901 A1
(43) Pub. Date: Mar. 5, 2009

(54) DETOXIFICATION METHOD

(76) Inventor: Russell Guy Ashley Jones, Potters Bar (GB)

Correspondence Address: FULBRIGHT & JAWORSKI, LLP, 1301 MCKINNEY, SUITE 5100, HOUSTON, TX, 77010-3095, US

(21) Appl. No.: 11/816,042

(22) PCT Filed: Feb. 10, 2006
(86) PCT No.: PCT/GB2006/000466

§ 371 Date: Dec. 10, 2007

(30) Foreign Application Priority Data
Feb. 11, 2005 (GB) 0502901.2

(51) Int. Cl. A61K 39/395; C07K 14/00; C07K 14/415; A61P 31/00; A61K 38/16; A61K 39/00

(52) U.S. Cl. 424/130.1; 530/350; 530/370; 514/12; 424/184.1

(57) ABSTRACT

The present invention provides a protein toxoid for use in a method of treating the human or animal body by therapy, wherein all cysteine residues in said protein toxoid which are free under reversibly denaturing conditions have been alkylated.

### FIELD OF INVENTION

The present invention relates to a method for inactivating toxins to produce protein toxoids, vaccines comprising the protein toxoids and methods of treating and preventing disease caused by toxins comprising administering the protein toxoids. The protein toxoids may also be used as laboratory reagents and in the production of therapeutic antitoxin.

### BACKGROUND TO THE INVENTION

Toxoids (inactivated toxins) have traditionally been produced by chemical inactivation or cross-linking using formaldehyde (Formalin) to principally react with the large number of lysine residues present in the toxin protein. For example, whilst up to 72 lysine residues of botulinum toxin A can be methylated without affecting toxicity, modification of 3 additional residues causes a precipitous loss of activity (Sathyamoorthy & DasGupta 1988). By contrast, botulinum A toxin contains only 9 cysteine residues, 4 of which are cross-linked by disulphide bonds. Type B and E toxins contain 10 and 8 cysteine residues respectively (Antharavally et al. 1998; Antharavally and DasGupta 1998).

Toxoids produced by formaldehyde treatment have been described previously and have been used as vaccines and in the production of antitoxins. The prompt administration of therapeutic antitoxin is currently the only specific treatment for botulism and tetanus. Current vaccines produced by formaldehyde inactivation of the toxin often give poor antibody responses in man.

Production of botulinum antitoxin, for example, typically involves immunising animals (either horses or sheep) with high doses of botulinum toxoid made by formaldehyde (Formalin) detoxification. Higher neutralising antibodies can be produced by boosting the animals with high doses of botulinum toxin. However, due to health and safety (and insurance) constraints it is now virtually impossible to immunise animals with the active toxin during the immunisation procedure.

Type B botulinum toxin can be only partially inactivated (13 fold reduction) by the prolonged alkylation of cysteine residues with iodoacetamide alone (Beers and Reich 1969). It has also been reported that botulinum type A toxin and tetanus toxin are either unaffected or have their toxicity reduced by up to 50% following treatment (Knox et al. 1970, Schiavio et al. 1990, de Pavia et al. 1993). An approximately 1,000 fold decrease in toxicity has been previously reported following reduction and alkylation or carboxymethylation of botulinum type A toxin (de Pavia et al. 1993). Reduction followed by formaldehyde (0.5%) treatment in the presence of a reducing agent has been demonstrated to cause a marked decrease in immulogenicity (production of neutralising antibody) relative to conventional toxoid produced by formaldehyde treatment alone (Sugiyama et al. 1974).

### SUMMARY OF THE INVENTION

The present inventor has surprisingly found that toxicity of botulinum toxin may be reduced to insignificant levels by alkylating cysteine residues in the toxin under reversibly denaturing conditions. This method of inactivating botulinum toxin does not cause gross conformational changes to the toxin molecule and so the new toxoid retains most of the immunogenicity of the original active toxin but without the risk of toxicity.

This method of alkylating cysteine residues under reversibly denaturing conditions may be used to inactivate other protein toxins. The present inventors have thus provided a mild method for toxin inactivation that is generally applicable to protein toxins to produce protein toxoids. The protein toxoids are useful in the production of therapeutic antitoxins, as vaccines and as safe laboratory reagents.

Accordingly, the present invention provides:
a protein toxoid for use in a method of treating the human or animal body by therapy, wherein all cysteine residues in said protein toxoid which are free under reversibly denaturing conditions have been alkylated;
a method for producing a protein toxoid according to the invention, which method comprises:
(i) alkylating cysteine residues in a protein toxin under reversibly denaturing conditions;
(ii) renaturing said protein;
(iii) optionally repeating steps (i) and (ii) one or more times; and
(iv) formulating the protein toxoid with a pharmaceutically acceptable diluent or carrier;
   a protein toxoid obtained or obtainable by a method according to the invention;
   a protein toxoid according to the invention for use in a method of treating the human or animal body;
   a pharmaceutical or vaccine composition comprising a protein toxoid according to the invention, together with a pharmaceutically acceptable carrier or diluent;
   use of a protein toxoid according to the invention in the manufacture of a medicament for treating or preventing bacterial infection;
   use of a protein toxoid according to the invention in the manufacture of a medicament for use in treating or preventing poisoning by a protein toxin;
   use of a protein toxoid according to the invention as a laboratory reagent;
   a method of vaccinating against a toxic bacteria, toxin or biological warfare agent comprising administering to a subject in need thereof, a protein toxoid according to the invention in an amount effective to induce an immune response against the toxoid;
   a method of treating a bacterial infection comprising administering to an individual in need thereof a therapeutically effective amount of a protein toxoid according to the invention;
   a method of treating an individual exposed to a toxic bacterium, toxin or a biological warfare agent, which method comprises administering to an individual in need thereof a therapeutically effective amount of a protein toxoid according to the invention;
   use of a protein toxoid according to the invention in the manufacture of a medicament for use in antitoxin production;
   a method of producing an antitoxin, which method comprises administering a protein toxoid according to the invention to an individual in an amount effective to induce anti-toxoid antibodies, taking blood from said individual and separating serum from the blood, wherein said serum comprises said antibodies;
   an antitoxin produced by a method according to the invention;
   use of an antitoxin according to the invention in the manufacture of a medicament for treating an individual exposed to a toxic bacterium, toxin or biological warfare agent; and
   a method of treating an individual exposed to a toxin, toxic bacterium or a biological warfare agent, which method comprises administering to an individual in need thereof a therapeutically effective amount of an antitoxin according to the invention.

### BRIEF DESCRIPTION OF THE FIGURES

FIG. 1 shows the results of chemical inactivation of botulinum toxin B following one (sample 1b), two (sample 2b) and three (sample 3b) rounds of alkylation under reversible denaturing conditions as determined using the isolated mouse phrenic nerve hemidiaphragm assay.

FIG. 2 shows the amount of protein left in solution following chemical inactivation of botulinum toxin B following two rounds of alkylation under reversible denaturing conditions in the presence of 150 mM, 300 mM or 1M NaCl.

FIG. 3 shows the inactivation of botulinum type B toxin in the presence of 0.5M, 1M and 2M NaCl. The local flaccid paralysis assay was used to determine activity.

FIG. 4 shows the amount of protein in solution following storage for two months at 4° C. and centrifugation of botulinum toxin B inactivated by one, two or three rounds of alkylation under reversible denaturing conditions in the presence of 0.5M, 1M or 2M NaCl.

FIG. 5 demonstrates the effect of pH on toxoid stability. The amount of protein in solution following storage at 4° C. for one month at pH5, pH6 or pH8 is shown.

FIG. 6 shows the activity of botulinum toxin B and botulinum toxoid B of the invention measured with the local flaccid paralysis assay (24 hours post injection).

FIG. 7 shows the endopeptidase activity of botulinum toxin B, toxoid of the invention, formaldehyde toxoid, buffer control and saline control. Endopeptidase activity was measured using HV50 substrate (assuming an initial 1 in 4 dilution of toxoid).

FIG. 8 shows the specific antibody levels detected by ELISA using mouse serum ten weeks after immunisation with 15 μg formaldehyde toxoid, 15 μg toxoid of the invention, 2 μg formaldehyde toxoid, 2 μg toxoid of the invention or normal mouse serum (NMS).

FIG. 9 shows the effect of immunisation with toxoid of the invention compared to formaldehyde toxoid. Neutralisation of botulinum toxin B by serum from immunised mice was determined using the local flaccid paralysis assay (24 hours post injection).

### DETAILED DESCRIPTION OF THE INVENTION

### Toxoid

A protein toxoid, or inactive toxin, of the invention is a protein toxin in which the cysteine residues have been alkylated under reversibly denaturing conditions. A protein toxoid of the invention is one wherein all cysteine residues which are free under reversibly denaturing conditions are alkylated. Free cysteine residues are typically those that are accessible to a cysteine alkylating agent under reversibly denaturing conditions, or cysteine residues that are not disulphide bonded. Typically, all free non-disulphide linked cysteine residues are alkylated but the cysteine residues which are disulphide bonded in the toxin may also be alkylated. In particular, all the cysteine residues which are disulphide bonded in the toxin may be alkylated where a reducing agent is used to denature any disulphide bonds present in the toxin from which the toxoid is derived.

The protein toxoid may be produced by a single incubation with an alkylating agent under denaturing conditions followed by a renaturation step. The protein toxoid may be produced by two or more cycles of alkylation under reversibly denaturing conditions and renaturation. The first round of denaturation, alkylation and renaturation results in a reduction in toxicity compared to unmodified toxin. Subsequent rounds may produce a pronounced further decease in toxicity. Accordingly, an inactive toxin preparation of the invention is one in which substantially all the toxin molecules have been inactivated.

Substantially all the toxin molecules in a toxoid preparation are typically inactivated where the preparation exhibits undetectable toxin activity or less than about ten thousand fold or about a million fold, for example about two, three, four, five, ten or a hundred million or a billion fold, of the toxicity of a preparation comprising an equivalent amount of unmodified toxin.

A toxoid of the invention may retain some endopeptidase activity, but without in vivo toxicity. Thus, the toxoid of the invention retains more of the original toxin's structure than formaldehyde toxoid which has no endopeptidase activity.

The alkylation step may be carried out with or without using a reducing agent to reduce any disulphide bonds in the toxin molecule.

For safety reasons, the protein toxoid may be tested to check for residual toxicity. Any suitably sensitive and fully functional model or models may be used to confirm that material is not toxic. For example, the isolated mouse phrenic nerve hemidiaphragm assay (Jones et al. 1999) or mouse local flaccid paralysis assay (Sesardic et al. 1996) may be used.

Protein toxoids produced by a method of the invention have negligible toxicity and exhibit essentially no reversal to native toxicity (at least about ten thousand fold less toxicity than native toxin) and so are safe for use as vaccines, therapeutic agents, laboratory reagents and in antitoxin production.

Protein toxoids of the invention may be used to generate neutralizing antibodies against the native toxin from which the toxoid is derived. Typically, immunization with a toxoid of the invention results in the production of higher levels of neutralizing antibody than immunization with formaldehyde toxoids.

Vaccines comprising an inactive toxin according to the invention may be used in the treatment or prevention of bacterial infection or in the treatment or prevention of the effects of toxins or biological warfare agents.

### Inactivation Method

The present invention provides a method for producing protein toxoids that are safe for use as immunogens. The method of inactivating a protein toxin to produce a protein toxoid comprises:
(i) alkylating cysteine residues in a protein toxin under reversibly denaturing conditions;
(ii) renaturing said protein; and
(iii) optionally repeating steps (i) and (ii) one or more times if required.

Steps (i) and (ii) may be carried out once or repeated once, more than once, for example two, three, four, five or ten times. A substantial reduction in toxicity may be obtained after a single incubation and in such cases no further repetition is needed. A substantial reduction in toxicity is preferably a complete inactivation. A toxin is completely inactivated where it demonstrates no detectable toxicity, for example, no detectable toxicity in the hemidiaphragm assay (Jones et al. 1999) or local flaccid paralysis assay (Seardic et al. 1996).

A toxoid having no detectable toxin activity of the unmodified toxin is considered, for the purposes of the present invention, as being an inactive toxin.

The inactivation method may include one or more reduction step. The reduction steps of the method of the invention may be carried out under any conditions suitable for reducing disulphide bonds. Typically, the toxin is exposed to a reducing agent. The reducing agent may be mixed with the toxin and incubated for from about 30 minutes to about 12 hours (overnight), for example for about 1, 2, 3 or 4 hours at a temperature of from about 4° C. to about 37° C., for example at about 20° C. (room temperature), 25° C., 30° C. or 35° C. Typically, reduction is carried out before alkylation but the reducing agent may be added at the same time as the alkylating agent.

The toxin may be dialysed against the reducing agent. Dialysis may be carried out at temperature of from about 4° C. to about 37° C., for example 20° C., 25° C., 30° C. or 35° C. for a period of from about 30 minutes to about 12 hours (overnight), for example for about 1, 2, 3 or 4 hours. The temperature may be shifted partway through the dialysis procedure. For example, dialysis may be carried out at room temperature for about 2 hours then at 37° C. for about 1 hour. The reaction mixture may be kept in the dark during the dialysis.

Any suitable reducing agent may be used. The reducing agent may be selected from dithiothreitol (DTT), 2-mercaptoethanol, glutathione, L-cysteine, TCEP-HCL and sodium borohydride. The reduction step may be carried out under reversibly denaturing conditions.

Cysteine residues in the toxin are alkylated. The allkylation step is carried out by adding an alkylation agent under conditions suitable for alkylation. Suitable alkylation agents include iodoacetamide, iodo acetic acid, N-ethylmaleimide and N-isopropyliodoacetamide. The alkylation step is typically carried out at a suitable pH at which effective alkylation takes place, but which does not adversely affect the protein. For example, iodoacetamide is optimally used at or around pH 8.0 and its effectiveness may be decreased at lower pH. Any suitable buffer may be used which is capable of effectively controlling the pH and which does not deleteriously react with the toxin, the alkylating agents or the alkylation process.

Alkylation is carried out under reversible denaturing conditions. A chaotropic agent such as urea and/or guanidine may be added during the alkylation step in order to denature the toxin. The chaotropic agent may be added to any suitable concentration, for example, 2M, 4M or 8M urea or guanidine may be used.

Typically, the reduced toxin is incubated with the alkylation agent for from about 30 minutes to about 12 to 14 hours (overnight), for example for about 1, 1½, 2, 3 or 4 hours, at a temperature of from about 4° C. to about 37° C., for example at about 20° C. The incubation may be carried out in the dark.

Excess alkylating agent may be removed and/or the protein renatured by dialysis, for example by dialysing the reaction mixture against Tris NaCl in the absence of any denaturing reagent. Dialysis against Tris NaCl may be carried out under any suitable conditions, typically against a first volume, for example, of approximately 3000 volumes (such as 1 litre) of Tris NaCl for two hours and then against a fresh aliquot, for example, of approximately 3000 volumes (such as 1 litre) of Tris NaCl overnight at 4° C. in the dark.

The use of inert stabilisers and chelating agents such as EDTA or EGTA may also be incorporated into the inactivation procedure.

After the final round of allylation, the reagents are removed from the reaction mixture and the toxoid is renatured, typically by dialysis with Tris NaCl as described above.

The resulting inactive toxin may be stored under any conditions under which it is stable, typically at about 4° C. in the dark.

The method may comprise a final chemical stabilisation step. Alternatively, the entire inactivation procedure may be performed in the presence of one or more suitable stabilising agent. The inactivated toxin may be stabilised by any suitable method, such as by a low concentration formaldehyde treatment. Stabilising or chelating agents may also be added before, during or after the inactivation method. Suitable stabilising agents include NaCl at high concentrations (for example from about 1M to about 4M, such as about 1.5M, 2M, 2.5M or 3M NaCl), sorbitol, manitol, dextrose, glycerol and gelatine. Suitable chelating agents include ethylenediaminetetraacetic acid (EDTA) and ethylenebis(oxyethylenenitrilo)tetraccetic acid (EGTA).

The method may further comprise formulating the toxin with a pharmaceutically acceptable carrier or diluent and optionally an adjuvant.

### Toxin

The toxin may be any protein toxin comprising one or more cysteine residue. The toxin may, for example, be an intracellularly acting toxin composed of two chains joined covalently by an inter-chain disulphide bond.

The toxin may be derived from any suitable organism. For example, the toxin may be derived from a plant. Suitable plant toxins include abrin and ricin.

The toxin may be an animal toxin, for example a snake toxin such as α-bungarotoxin, β-bungarotoxin, cobratoxin, crotoxin, erabutoxin, taicatoxin or textilotoxin, a spider toxin such as agatoxin, atracotoxin, grammotoxin, latrotoxin, phoneutriatoxin, plrixotoxin and versutoxin or a scorpion toxin such as margatoxin, iberiotoxin or noxiustoxin.

The toxin is preferably a bacterial toxin, such as a Clostridial neurotoxin, for example botulinum toxin or tetanus toxin, diphtheria toxin, cholera toxin, Bordetella pertussis toxin, Pseudomonas endotoxin A, shiga toxin, shiga-like toxins and E. coli heat labile toxin (HLT). Where the toxin is a botulinum toxin it may be type A, B, C, D, E, F or G.

The shiga-like toxin may be one produced by E. coli. The bacteria from which the toxin is derived is typically one involved in causing disease, such as Clostridiuin tetani, Clostridium botulinum, Corynebacteriun diphtheriae, Escherichia coli, Vibrio cholerae, Shigella dysenteriae, Bordetella pertussis or Pseudomonas aeruginosa. The toxin, or bacteria from which the toxin is derived, may be one which is used as a biological warfare agent.

Toxins for use in the preparation of toxoids may be obtained by any suitable method. Such methods are well known in the art. A bacterial toxin for use in a method of the invention may typically be obtained from cultured bacteria. For example, tetanus toxin may be obtained from a culture of Clostridium tetani, botulinum toxin from a culture of Clostridium botulinum, diphtheria toxin from a culture of Corynebacterium diphtheriae, heat labile factor from a culture of Escherichia coli, cholera toxin from a culture of Vibrio cholerae, pseudomonas exotoxin A from a culture of Pseudomonas aeruginosa and shiga toxins from a culture of Escherichia coli or Shigella dysenteriae. Methods for extracting toxins from bacterial cultures are well known in the art.

Plant toxins for use in a method of the invention may be obtained from suitable plant material, such as beans or seeds, according to known methods. For example, ricin may be obtained from Ricinus communis beans and abrin from the seeds of Abrus precatorius.

The toxin used in a method of the invention is preferably in isolated form. The toxin may be mixed with carriers or diluents. For example, the toxin may be mixed with a sodium phosphate, sodium chloride, pH 7.4 buffer. The toxin, such as botulinum toxin, may be complexed with or associated with other non-toxic proteins. Protective proteins such as gelatin may, in some instances, interfere with the reduction of disulphide bonds in the toxin and so generally the toxin will be free from protective proteins such as gelatin at least for the initial activation step. In later steps of inactivation method, gelatin may be added as a stabilisation agent. The toxin may be in a substantially purified form. A toxin in substantially purified form is generally at least about 70%, for example at least about 80%, 90%, 95%, 98% or 99% free from other bacterial or plant components.

### Laboratory Reagent

Protein toxoids of the invention are useful as laboratory reagents. For example, a toxoid of the invention is useful in a method of inducing or selecting for anti-toxin monoclonal antibodies. Methods for inducing and selecting monoclonal antibodies are well known in the art.

Laboratory uses of the toxoids of the invention also include their use in immunoassays. Various suitable immunoassay techniques are known in the art.

### Vaccines

The present invention provides a vaccine composition comprising protein toxoid comprising alkylated cysteine residues obtainable by a method of the invention, a pharmaceutically acceptable carrier or diluent and optionally an adjuvant. Also provided is the use of a toxoid of the invention in the manufacture of a vaccine for the prevention of a bacterial infection or a vaccine for protection against a toxin or biological warfare agent.

A vaccine of the invention may be administered to an individual in need thereof in an amount effective to induce an immune response to the toxin from which the toxoid is derived. The immune response may be an antibody response or a T-cell response to the toxin. On subsequent exposure to the toxin, an immune response to the toxin is elicited in a vaccinated individual against the toxin. The vaccinated individual is then protected against infection by bacteria which express the toxin or against a biological warfare agent which comprises the toxin or bacteria which express the toxin.

An individual in need of vaccination is typically an individual who has no active immunity against the toxin. The vaccine may therefore be administered to a baby or infant. Alternatively, the vaccine may be given to an older child or adult as a booster vaccine, where the individual has previously been vaccinated or otherwise exposed to the toxin. The individual may be at risk of exposure to the toxin or bacteria which expresses the toxin. The vaccine may also be administered to an individual who it is thought may recently have been exposed to the bacteria which expresses the toxin, for example where the toxin is tetanus toxin and the individual has an open wound or where the individual has been in contact with another individual known to be infected with bacteria which express the toxin.

Where the vaccine is used to protect against biological warfare agents, the individual in need of vaccination is typically an individual living or working in or close to a war zone. For example, the individual may be a member of the armed forces.

The individual in need of vaccination may be an individual who may be brought into contact with bacteria which express the toxin, or with active toxin as a result of that work. The individual may be a laboratory worker, a veterinarian, or a health worker such as a nurse or a doctor.

A vaccine composition of the invention may comprise one or more toxoids of the invention optionally in combination with one or more other active vaccine component. For example, tetanus toxoid of the invention may be combined with diphtheria toxoid of the invention and/or the tetanus/diphtheria toxoids of the invention may be combined with whole-cell or acellular pertussis vaccine (for example, inactivated pertussis toxin according to the present invention), inactivated poliovirus vaccine, trivalent oral poliovirus vaccine or Haemophilus influenzae type b (Hib) conjugate vaccine. Such combination vaccines are particularly suitable for administration to babies or infants.

### Therapy

Toxoids according to the invention are also useful in the therapeutic treatment of bacterial infections. Accordingly, the present invention provides a pharmaceutical composition comprising a toxoid which comprises alkylated cysteine residues together with a pharmaceutically acceptable carrier or diluent. Also provided is the use of a toxoid of the invention in the manufacture of a medicament for treating an individual exposed to a biological warfare agent.

The bacterial infection may be botulism, tetanus, diphtheria, cholera or a Pseudoinonas, Shigella or E. coli infection or an infection caused by any other toxic bacterium. The bacterial infection may be caused by entry of the bacteria into the blood or other internal organ or may be an infection of the skin or alimentary canal (such as botulism or E. coli food poisoning). The biological warfare agent may be botulinum toxin, ricin, abrin or any other agent utilising the toxic effect of a protein toxin.

A pharmaceutical composition of the invention may comprise one or more toxoid of the invention, for example ricin, abrin and/or botulinum toxoid. The pharmaceutical composition may additionally comprise one or more antibiotic, such as doxycycline, ciprofloxacin, penicillin, rifampin, vancomycin, clindamycin, claritliromycin or amoxicillin.

Toxoids of the invention may be used to block entry of active toxin produced by infecting bacteria and so administration of a pharmaceutical composition of the invention to an individual in need thereof preferably takes place as soon as possible after exposure of the individual to the infectious agent or toxin. Pharmaceutical compositions of the invention may also be used to boost the individual's immune response to the infecting bacteria or toxin.

A method of treating a bacterial infection, toxin poisoning, such as E. coli HLT poisoning or botulism, or the effects of biological warfare agents provided by the invention comprises administering to an individual in need thereof a therapeutically effective amount of toxoid of the invention. A therapeutically effective amount is an amount which blocks entry of active bacterial toxin into cells of the individual and/or which stimulates an immune response to the toxin.

An individual in need of treatment is one which has been recently exposed to a bacteria which expresses the toxin or to the toxin, for example to a biological warfare agent comprising the toxin or a bacteria which expresses the toxin, wherein the toxoid is derived from said toxin. Preferably the inactive toxin is administered to the individual as soon as possible after exposure to the toxin, bacteria or agent.

### Antitoxin Production

Toxoids of the present invention are useful in antitoxin production. Accordingly, the present invention provides a method of producing an antitoxin comprising administering a toxoid which comprises alkylated cysteine residues to an individual in an amount effective to induce anti-toxin antibodies, taking blood from the individual and separating serum from the blood. Anti-toxin antibodies are present in the serum (or plasma) and may, optionally be purified from the serum. The individual may be a human or animal, such as a horse, cow, sheep or goat. Where the individual is a non-human animal, the method may comprise sacrificing the animal. Antitoxin antibodies may be purified from the serum. The antibodies may be processed to produce anti-toxin antibody fragments such as F(ab′)2, Fab′, Fab or Fv. An antitoxin of the invention may be produced from the donated blood or plasma of humans following a course of vaccination with an inactivated toxin of the invention.

Antitoxins produced by such a method of the invention may be used in the treatment of bacterial infection by a bacteria expressing the toxin and also in the treatment of individuals exposed to toxin, for example to biological warfare agents comprising the toxin or bacteria which express the toxin. The antitoxin is preferably administered to an individual immediately following clinical diagnosis of the bacterial infection or as soon as possible after exposure to the agent or bacteria.

### Administration

Formulation with standard pharmaceutically acceptable carriers and/or diluents and/or stabilisers may be carried out using routine methods in the pharmaceutical art. For example, the toxoid may be dissolved in physiological saline or water for injections. The exact nature of a formulation will depend upon several factors including the desired route of administration. Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

The toxoid is typically administered by a parenteral route. The inactive toxin may be administered subcutaneously, intravenously, intramuscularly, intraperitoneally, intrasternally, transdermally or by infusion techniques. Subcutaneous or intramuscular administration typically require use of an adjuvant. Subcutaneous or intramuscular administration may be used to boost following initial administration of the vaccine via an intravenous or intraperitoneal route in order to reduce the risk of anaphylactic shock.

Vaccines and pharmaceutical compositions may be prepared from one or more of the toxoids and/or antitoxins defined herein and a physiologically acceptable carrier or diluent. Typically, such vaccines are prepared as injectables, either as liquid solutions or suspensions; solid forms suitable for dissolving in, or suspending in, liquid prior to injection may also be prepared. The preparation may also be emulsified, or the protein encapsulated in a liposome. The active immunogenic ingredient may be mixed with a carrier or diluent which is pharmaceutically acceptable and compatible with the active ingredient. Suitable carriers and diluents are, for example, sterile water, saline, phosphate buffered saline (PBS), dextrose, glycerol, ethanol, or the like and combinations thereof. Preferably the vaccine or pharmaceutical composition is in the form of a sterile, aqueous, isotonic saline solution. A carrier protein may be used if desired.

In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of anti-toxin antibodies resulting from administration of the toxoid in vaccines which are also comprised of the adjuvant.

The vaccines are conventionally administered parentally, by injection, for example, either subcutaneously, intracutaneously or intramuscularly. The vaccines may alternatively be administered by local administration to the skin, such as by intracutaneous or subcutaneous administration. Additional formulations which are suitable for other modes of administration include suppositories, oral formulations and formulations for transdermal administration. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. a suspension. Reconstitution is preferably effected in buffer.

Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

Vaccine compositions suitable for delivery by needleless injection, for example, transdermally, may also be used.

The dose may be determined according to various parameters, especially according to the age, weight and condition of the patient to be treated; the route of administration; and the required regimen. A physician will be able to determine the required route of administration and dosage for any particular patient.

The vaccines are administered in a manner compatible with the dosage formulation and in an amount that will be prophylactically and/or therapeutically effective. The quantity to be administered, which is generally in the range of 5 μg to 100 mg, preferably 250 μg to 10 mg, more preferably from 1 mg to 3 mg, of antigen per dose, depends on the subject individual to be treated, capacity of the individual's immune system to synthesize antibodies, and the degree of protection desired. Precise amounts of active ingredient required to be administered may depend on the judgement of the practitioner and may be peculiar to each individual.

The vaccine may be given in a single dose schedule, or preferably in a multiple dose schedule. A multiple dose schedule is one in which a primary course of vaccination may be 1 to 10 separate doses, for example 3, 4, 5, 6, 7 or 8, followed by other doses given at subsequent time intervals required to maintain and or reinforce the immune response, for example at 1 to 4 months for a second dose, and if needed, a subsequent dose(s) after several months. The dosage regimen will also, at least in part, be determined by the need of the individual and be dependent upon the judgement of the practitioner.

An exemplary, non-limiting range for a therapeutically or prophylactically effective amount of an inactivated toxin of the invention is from 1 ng/kg to 20 mg/kg, for example from 1 μg/kg to 10 mg/kg, 10 μg/kg to 1 mg/kg. The composition can be administered by intravenous infusion at a rate of less than 30, 20, 10, 5 or 1 mg/min to reach a dose of about 1 to 100 mg/m² or about 5 to 30 mg/m². Dosage values may vary with the severity of the condition of the individual.

Antitoxins of the invention may be administered in a dose of from about 10U to about 500,000U, for example about 20,000U, about 10,000U or about 1,000U. The exact dose may be determined empirically by a physician. Antitoxin may be administered intramuscularly or intravenously or, where the antitoxin is for treatment of tetanus, may be injected directly into the wound. Where the antitoxin is a horse, or other animal serum, the patient may be tested for sensitivity using a skin test prior to administration. Where the skin test indicates sensitivity, the individual may be desensitized with dilute antitoxin given in graduated doses with appropriate emergency supportive measures.

The present invention is described with reference to the following, non-limiting Examples:

### EXAMPLES

### Example 1

### Inactivation of Botulinum Type B Toxin

B toxin (100 μl, 1 mg/ml, 8×10⁷ LD₅₀/ml) was mixed with an equal volume (100 nl) of Tris buffer (50 mM Tris, 1 mM EDTA, 0.9% NaCl, pH 8.0) containing either (a) 0.0M, or (b) 2M urea. This material (200 μl) was then mixed with an equal volume (200 μl) of 250 mM Iodoacetamide in Tris buffer containing either (a) 0 or (b) 2M urea and incubated for 90 minutes at room temperature (RT) in the dark. The material was then dialysed (10 kDaMWCO) against cold Tris buffer containing 0M urea in the dark at 4° C. Samples (˜50 μl) 1a and 1b were removed and stored at 4° C. Remaining material was then dialysed against 250 ml of 125 mM, Iodoacetamide in Tris buffer, containing either (a) 0.0M, or (b) 2M urea in the dark for 3 h at RT. Followed by dialysis against cold Tris buffer containing 0M urea in the dark at 4° C. Samples (˜50 μl) 2a and 2b were removed and stored at 4° C. The procedure was repeated once more and the final material 3a and 3b recovered.

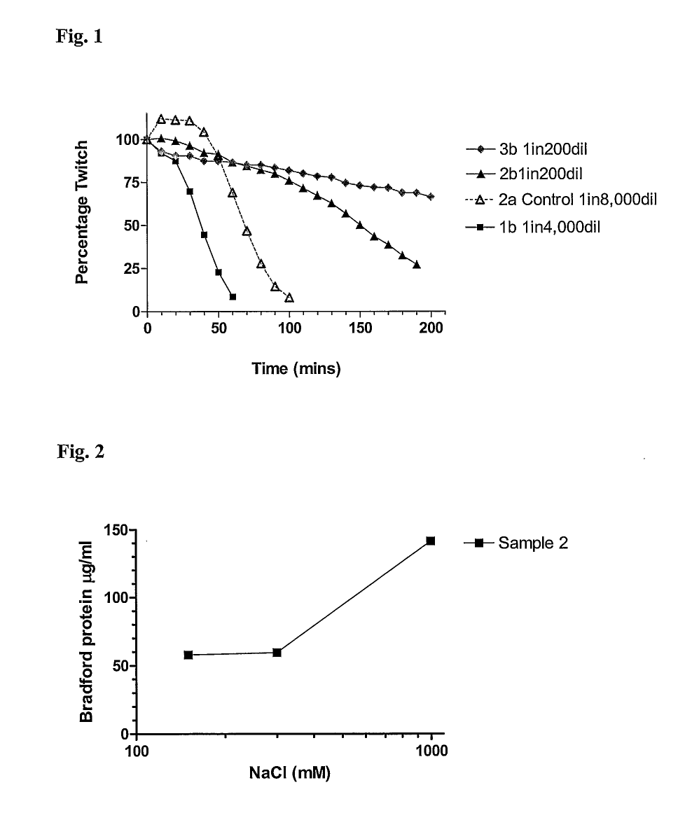

Residual toxicity was tested using the isolated mouse phrenic nerve hemidiaphragm assay. Briefly the assay was performed as described previously (Jones et al. 1999) using Krebs (NaCl 118 mM, KCl 4.83 mM, KH₂PO₄ 1.19 mM, NaHCO₃ 25 mM, MgSO₄.7H₂O 1.2 mM, D-glucose 11.1 mM, CaCl₂.2H₂O 2.54 mM) containing 0.2% gelatin, and the nerve stimulated at 1 Hz, 0.2 ms with a 6 ml tissue bath at 37° C. The results are shown in FIG. 1.

### Example 2

### Effect of Salt on Botulinum Toxin B Stability

B toxin (100 μl, 1 mg/ml, 8×10⁷ LD₅₀/ml) was mixed with an equal volume (100 μl) of Tris buffer (50 mM Tris, 1 mM EDTA, pH 8.0). This material (200 μl) was then mixed with an equal volume (200 μl) of 250 mM Iodoacetamide in Tris buffer containing 4M urea and either (a) 300 mM, (b) 600 mM, or (c) 2000 mM NaCl, mixed and incubated for 4 h at RT in the dark. The material was then dialysed (10 kDaMWCO) against cold Tris buffer containing either (a) 150 mM, (b) 300 mM, or (c) 1M NaCl in the dark at 4° C. Samples (˜50 μl) 1a, b and c were removed and stored at 4° C. Remaining material was then dialysed against 250 ml of 125 mM, Iodoacetamide in Tris buffer, containing 2M urea and either (a) 150 mM, (b) 300 mM, or (c) 1M NaCl in the dark for 3 h at RT. Followed by dialysis against cold Tris buffer containing 0M urea in the dark at 4° C. Samples 2a, b, c were removed and stored at 4° C.

Residual soluble protein was estimated by the Bradford protein assay after centrifuging the samples (Coomassie Plus™). The results are shown in FIG. 2.

### Example 3

### Effect of Salt and Repeated Alkylation on Botulinum Toxin B Inactivation

One volume of botulinum type B toxin (100 μl, 1 mg/ml or ˜6.7 μM containing 8×10⁷ LD50 U/ml) was mixed within a Class 1 safety cabinet with three volumes (300 μl) of freshly prepared alkylating agent (66.67 mM Tris, 1.33 mM EDTA, 2.67M urea, NaCl pH8.0 containing 266.7 mM freshly dissolved Iodoacetamide, and either (a) 666.7 mM, (b) 1,333 mM or (c) 2667 mM NaCl), and incubated for 3 h at 37° C. in the dark.

The material was then dialysed against cold Tris buffer pH8.0 containing either (a) 500 mM, (b) 1M or (c) 2M NaCl (1L X2, overnight in the dark at 4° C.) utilising a 10 kDaMWCO dialysis membrane. Samples 1a, b and c (each ˜50 μl) were removed and stored at 4° C.

The remaining material was then dialysed against 250 ml of 125 mM, Iodoacetamide in 50 mM Tris, containing either (a) 0.5M, (b) 1M, or (c) 2M NaCl in the dark for 3 h, stirring at RT (250 ml). This was followed by dialysis against cold Tris buffer pH8.0 containing either (a) 500 mM, (b) 1M or (c) 2M NaCl (1L X2, overnight in the dark at 4° C.). Samples 2a, b and c (each ˜50 μl) were removed and stored at 4° C. This procedure was repeated and the remaining material removed, (samples 3a, b and c) and stored at 4° C.

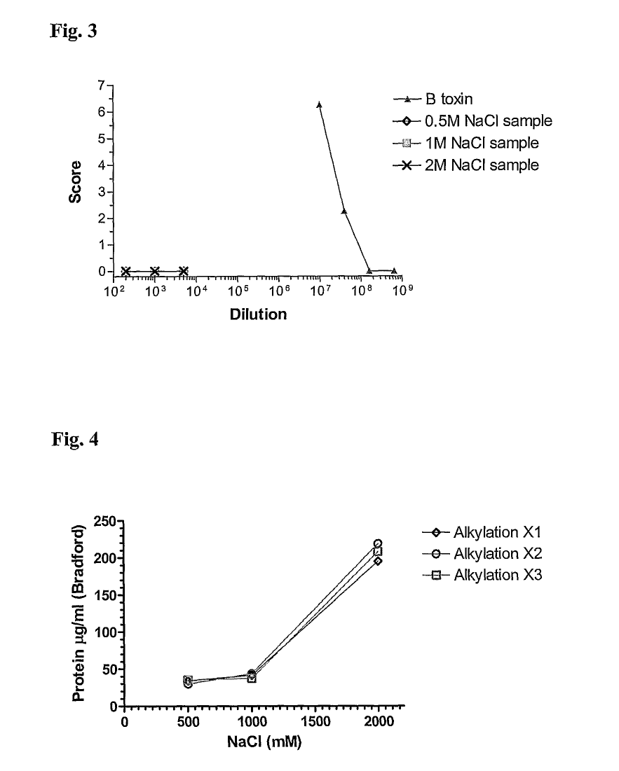

It was found that high concentrations of NaCl did not adversely affect toxin inactivation (FIG. 3) as tested using the local flaccid paralysis assay. High concentrations did, however, improve the stability of the toxoid (FIG. 4) as determined using the Bradford protein assay.

### Example 4

### Effect on pH on Stability of Botulinum Toxoid B

One volume of botulinum type B toxin (75 μl, 1 mg/ml or ˜6.7 μM containing 8×10⁷ LD50 U/ml) was mixed within a Class 1 safety cabinet with three volumes (225 μl) of freshly prepared alkylating agent (66.67 mM Tris, 1.33 mM EDTA, 2667 mM NaCl, 2.67M urea, pH8.0 containing 266.7 mM freshly dissolved Iodoacetamide), and incubated for 3 h at 37° C. in the dark.

The material was then dialysed against either cold 50 mM Tris buffer pH8.0, or 150 mM phosphate buffer pH 6.0 or 150 mM phosphate buffer pH5.0 containing 2M NaCl (3×μL) utilising a 10 kDaMWCO dialysis membrane. The resulting toxoid was removed and stored at 4° C. Samples were tested to confirm the loss of toxicity and, following storage at different pHs for one month at 4° C., for stability. A larger amount of precipitate was formed at pH 5 compared to pH 6 and 8 (FIG. 5).

### Example 5

### Immunisation Using Botulinum Toxoid B

One volume of botulinum type B toxin (750 μl, 1 mg/ml or ˜6.7 μM containing 8×10⁷ LD50 U/ml) was mixed within a Class 1 safety cabinet with three volumes (2,250 μl) of freshly prepared alkylating agent (66.67 mM Tris, 1.33 mM EDTA, 2667 mM NaCl, 2.67M urea, pH8.0 containing 266.7 mM freshly dissolved Iodoacetamide), and incubated for 3 h at 37° C. in the dark.

The material was then dialysed against phosphate buffer (3×μL, 150 mM Na₂HPO₄.2H₂O, 2M NaCl, pH7.0 at RT) utilising a 10 kDaMWCO dialysis membrane and the resultant toxoid removed and stored at 4° C.

### Hemidiaphragm Assay

Following inactivation a small amount of sample was suitably diluted to a safe concentration assuming limited inactivation before initial testing on the isolated phrenic nerve hemidiaphragm preparation (as outlined previously) and then if negative at suitably higher concentrations (up to 1 in 200 dilution). No toxicity was evident with the hemidiaphragm assay (data not shown) so the more sensitive in vivo local flaccid paralysis toxin assay was utilised.

### Local Flaccid Paralysis Assay

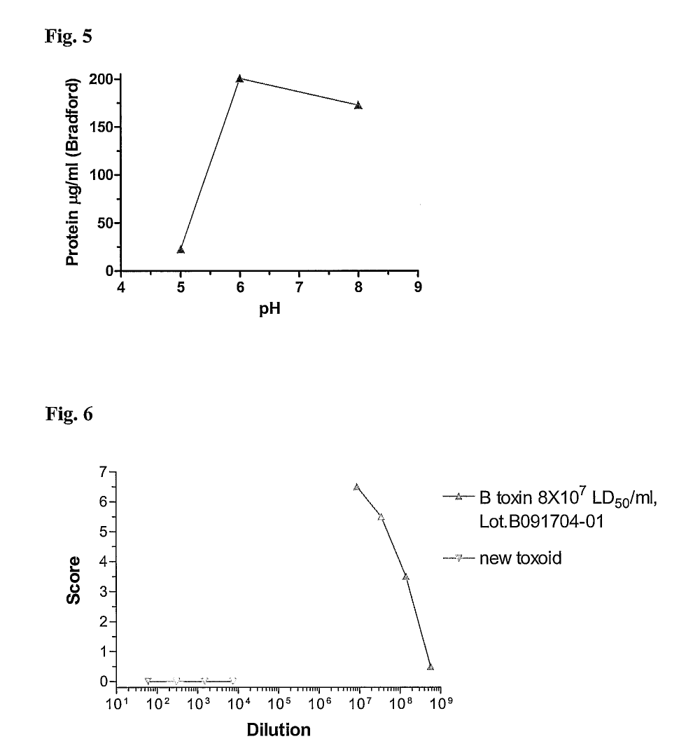

The procedure is based on the method of Takahashi et al. (1990; Sesardic et al. 1996). Briefly, female out-bred white mice (MF1, Harlan) were injected subcutaneously (SC) with 0.1 ml of diluted toxin B or inactivated material in the left inguinocrural region. Animals were independently scored by 2 or more trained scorers for the degree of abdominal flaccid paralysis or size of bulge/ptosis formed from 0 to 4. All dilutions were performed in gelatine phosphate buffer (0.2% w/v gelatine, 50 mM di-sodium hydrogen orthophosphate, pH6.5). No signs of toxicity were found with the inactivated material (see FIG. 6) indicating a greater than or equal to ˜40 million fold reduction in toxicity.

The toxoid material was centrifuged to remove any precipitate and a bicinchoninic acid (BCA) protein assay performed on the soluble material relative to a bovine serum albumin (BSA) standard curve. A correction factor of 0.63 was calculated using three batches of formaldehyde inactivated B-toxoid of known concentration on the BCA assay. From this an approx 67% recovery was calculated for the new toxoid.

### Endopeptidase Activity

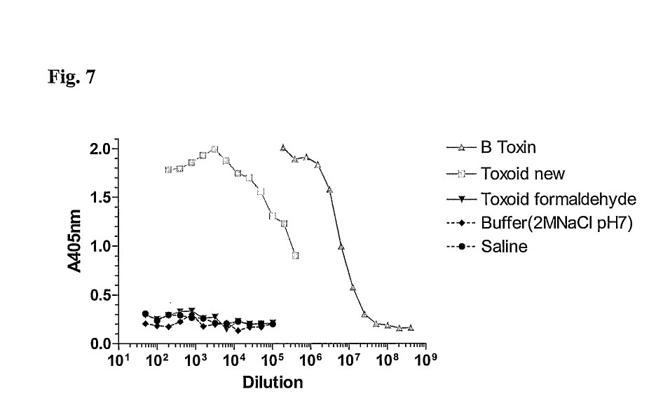

Sulfhydryl binding plates (96 well, Costar, C2509) were coated with 0.5 μg/well (10 g/ml) synthetic VAMP peptide (HV50 Cys; 46-94aa, or HV35Cys; 60-94aa) substrate in phosphate buffered saline (PBS) pH6.5 for 3 h at 37° C., blocked with PBS-Tween containing 5% skimmed milk powder for 2 h and washed with distilled water. Toxin or toxoid was reduced in the presence of 10 mM DTT in assay buffer (50 mM HEPES, 20 mM NaCl pH7 with 20 μM ZnCl₂) for 15-30 minutes prior to addition to the plate and finally diluted in assay buffer on the plate and incubated overnight at 37° C. before washing. Cleaved substrate specific (VAMP 77-84aaCys-KLH) rabbit antiserum was added at a 1 in 200 dilution and incubated at 37° C. for 90 min, then washed. Plates were incubated with goat anti-rabbit-HRP conjugate (Amdex-enzyme conjugate; Amersham) at 1 in 20 k at 37° C. for 90 min. After washing, plates were developed with ABTS/H₂O₂ substrate and the OD measured at 405 nm. No endopeptidase activity was evident in either formaldehyde inactivated toxoid, buffer or saline controls. However, the new toxoid retained a large amount of the original toxin's endopetidase activity (see FIG. 7), despite having no neurotoxic activity (FIG. 6).

### Toxoid Immunisation

Female BALB/c mice (6-8 weeks old) were immunised subcutaneously with 0.1 ml at each of two sites. Immunogen was mixed extensively with an equal volume of adjuvant to form a stable water in oil emulsion. Freund's complete adjuvant was used for the primary immunisation and Freund's incomplete adjuvant for all re-immunisations. Groups of five were immunised with either 2 μg formaldehyde toxoid (Group 1, low dose) or 2 μg new toxoid (Group 2, low dose) at weeks 0, 4 and 8, or with 15 μg formaldehyde toxoid (Group 3, high dose) or 15 μg new toxoid (Group 4, high dose) at weeks 0 and 4 only. Blood samples were taken two weeks after each immunisation, with a terminal bleed at week 10 and serum separated. Some of the material was then pooled for each group of 5 mice using equal volumes.

### ELISA

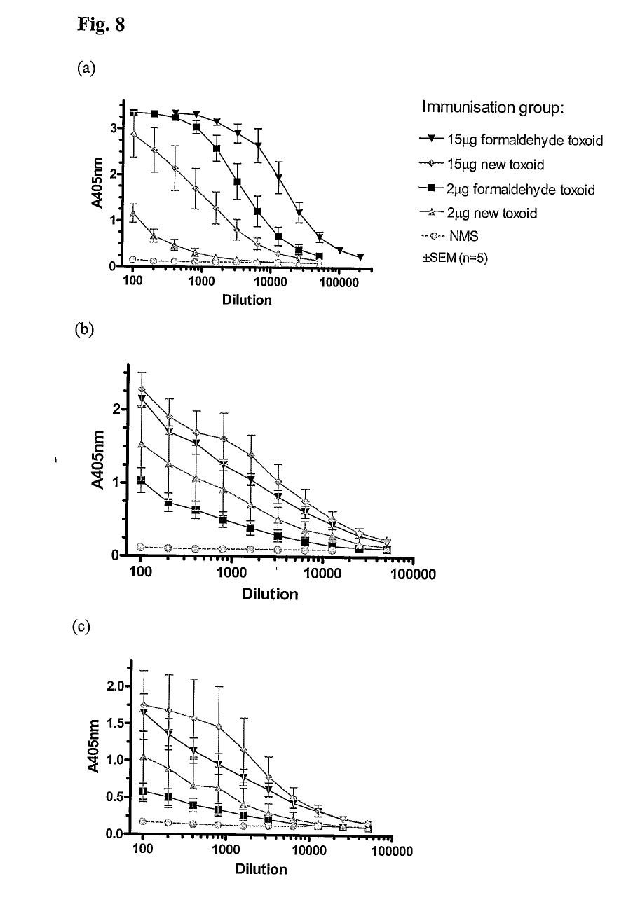

Botulinum type B toxin coated plates were purchased from Metabiologics Inc. Toxoid (1 μg/ml) was coated on 96 well plates (Nunc-Maxisorb) in carbonate buffer pH9.6 for 3 h at 37° C., blocked with skimmed milk powder (Marvel, 5%) in ELISA wash buffer (milk-EWB), for 2 h at 37° C. and washed with EWB (X5). Samples were diluted with milk-EWB and incubated for 90 minutes at 37° C. washed and HRP labelled anti-mouse IgG (Sigma, A9044) added at a 1 in 5,000 dilution, incubated, washed and ABTS substrate solution added. After approximately 30 minutes, plates were shaken and read at 405 nm. Specific binding antibody levels were highest against their respective toxoid used for immunisation (FIGS. 5a and b). Much higher levels of antibody were produced with the formaldehyde inactivated toxoid that were specifically detected using the formaldehyde inactivated toxoid coated plates (FIG. 8a). Relative amounts of binding to toxin coated plates (FIG. 8c), however, closely resembled binding to the new toxoid coated plates (FIG. 8b) indicating that the new toxoid is antigenically more similar to the native toxin.

### Neutralising Antibody

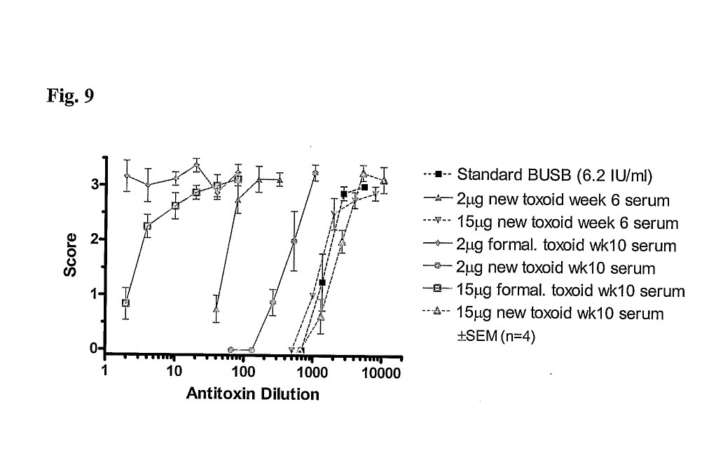

Neutralising antibodies were assessed using the local flaccid paralysis assay as described above with the following modifications. A fixed concentration of B toxin which produced a near maximal local flaccid paralysis was chosen. The second botulinum type B antitoxin International Standard (BUSB) was run in parallel in each assay and toxin was used at a final concentration of approximately 6 LD₅₀ U/ml. Negative (GPB) and positive (toxin) controls were included with each assay, and toxin/antitoxin, mixed and allowed to stand at RT for 1 h prior to injection. Using the principals of Probit parallel line analysis the activities relative to the International Standard were determined (FIG. 9). A significantly higher amount of neutralising antibody was generated with the new toxoid that was some 600 times greater than that produced with the formaldehyde inactivated toxoid (Table 1).

TABLE 1
Calculating toxin neutralising levels expressed in International Units (1 IU
by definition will neutralise ~10,000 toxin LD₅₀)

| Week | Immunogen Group | IU/ml serum | 95% confidence limits |
|---|---|---|---|
| 6 | 2 μg new toxoid | 0.2 | 0.16-0.27 |
| 6 | 15 μg new toxoid | 5.1 | 4.0-6.5 |
| 10 | 2 μg formaldehyde toxoid | Undetectable | |
| 10 | 2 μg new toxoid | 1.5 | 1.2-1.9 |
| 10 | 15 μg formaldehyde toxoid | 0.013 | 0.011-0.016 |
| 10 | 15 μg new toxoid | 8.0 | 6.3-10.0 |

### REFERENCES

Antharavally et al. (1998), Status of cys residues in the covalent structure of botulinum neurotoxin type A, B and E, J. Protein Chem. 17:187-196.
Antharavally and DasGupta, (1998), Covalent structure of botulinum neurotoxin type B; location of sulfhydryl groups and disulfide bridge and identification of C-termni of light and heavy chains, J. Protein Chem. 17:417-428.
Beers and Reich (1969), Isolation and characterization of Clostridium botulinum type B toxin. J. Biol. Chem. 244 (16), 4473-4479.
Jones et al. (1999), The effects of specific antibody fragments on the ‘irreversible’ neurotoxicity induced by Brown snake (Pseudonaja) venom, Br. J. Pharmacol. 126:581-584.
Knox et al. (1970). The role of sulfhydryl groups in the activity of type A botulinum toxin. Biochim. Biophys. Acta 214 (2), 350-354.
de Pavia et al. (1993), A role for the interchain disulfide or its participating thiols in the internationalisation of botulinum neurotoxin A revealed by a toxin derivative that binds to ecto-acceptors and inhibits transmitter release intracellularly, J. Biol. Chem. 268:20838-30844.
Sathyamoorthy and DasGupta, (1998), Reductive methylation of lysine residues of botulinum neurotoxin types A and B, Molecular and Cellular Biochemistry 83:65-72.
Schiavo, G., Papini, E., Genna, G., Montecucco, C., (1990). An intact interchain disulfide bond is required for the neurotoxicity of tetanus toxin. Infect. Immun. 58 (12), 4136-4141.
Sesardic et al. (1996), Refinement and validation of an alternative bioassay for potency testing of therapeutic botulinum type A toxin, Pharmacol Toxicol. 78:283-288.
Sugiyama et al (1974). Disulfide-immunogenicity relationship of botulinal toxins. Proc. Soc. Exp. Biol. Med. 145 (4), 1306-1309.
Takahashi et al. (1990). Assay in mice for low levels of Clostridium botulinum toxin. Int. J. Food Microbiol. 11 (3-4), 271-277.

1. A protein toxoid wherein all cysteine residues in said protein toxoid which are free under reversibly denaturing conditions have been alkylated.

2. A toxoid according to claim 1, which is derived from a bacterial toxin.

3. A toxoid according to claim 2, wherein the bacterial toxin is selected from the group consisting of diphtheria toxin, botulinum toxin, tetanus toxin, pseudomonas exotoxin A, shiga toxins, cholera toxin, and E. coli heat labile toxin (HLT).

4. A toxoid according to claim 1, which is derived from plant toxin.

5. A toxoid according to claim 4, wherein the plant toxin is ricin or abrin.

6. A toxoid according to claim 1, which is derived from a snake, spider or scorpion venom.

7. A method for producing a protein toxoid according to claim 1, which method comprises:
(i) alkylating cysteine residues in a protein toxin under reversibly denaturing conditions;
(ii) renaturing said protein;
(iii) optionally repeating steps (i) and (ii) one or more times; and
(iv) formulating the protein toxoid with a pharmaceutically acceptable diluent or carrier.

8. A method according to claim 7, wherein said cysteine residues are alkylated by incubating the protein toxin with an alkylating reagent selected from iodoacetamide, iodoacetic acid, N-ethylmaleimide or N-isopropyliodoacetamide.

9. A method according to claim 7, wherein said protein toxin is denatured using urea and/or guanidine.

10. A method according to claim 8, wherein the alkylating agent is removed in step (ii).

11. (canceled)

12. (canceled)

13. A pharmaceutical or vaccine composition comprising a protein toxoid according to claim 1, together with a pharmaceutically acceptable carrier or diluent.

14. A vaccine composition according to claim 13, which further comprises an adjuvant.

15. A method of treating or preventing bacterial infection or poisoning by toxic bacteria, a protein toxin, or a biological warfare agent, said method comprising administering to an individual in need thereof a therapeutically effective amount of a protein toxoid according to claim 1.

16. A method according to claim 15, wherein the bacterial infection is one or more of diphtheria, botulism, tetanus, cholera, E. coli heat labile toxin food poisoning, Pseudomonas infection or E. coli infection.

17. (canceled)

18. A method according to claim 17 wherein said poisoning is E. coli heat labile toxin food poisoning or botulism.

19. A method according to claim 17, wherein said poisoning is caused by a biological warfare type agent.

20. A method according to claim 17, wherein the protein toxoid is derived from botulinum toxin, ricin or abrin.

21. (canceled)

22. (canceled)

23. (canceled)

24. (canceled)

25. (canceled)

26. (canceled)

27. A method of producing an antitoxin, which method comprises administering a protein toxoid according to claim 1 to an individual in an amount effective to induce anti-toxoid antibodies, taking blood from said individual and separating serum from the blood, wherein said serum comprises said antibodies.

28. A method according to claim 27, wherein said individual is an animal.

29. A method according to claim 27, wherein said method further comprises sacrificing the animal.

30. A method according to claim 27, wherein said antibodies are purified from said serum.

31. A method according to claim 27, wherein said antibodies are processed to produce anti-toxin antibody fragments.

32. An antitoxin produced by a method according to claim 27.

33. (canceled)

34. A method of treating an individual exposed to a toxin, toxic bacterium or a biological warfare agent, which method comprises administering to an individual in need thereof a therapeutically effective amount of an antitoxin according to claim 32.
